(19) United States
(12) Patent Application Publication
(10) Pub. No.: US 2026/0137785 A1
(43) Pub. Date: May 21, 2026

(54) Compositions And Methods Of Treatment With Alternating Electric Fields And CD137 Agonists

(71) Applicants: NOVOCURE GMBH, Baar (CH); The Regents of the University of New Mexico, Albuquerque, NM (US)

(72) Inventors: Sara Piccirillo, Albuquerque, NM (US); Ayelet Orbach, Haifa (IL)

(21) Appl. No.: 19/395,302

(22) Filed: Nov. 20, 2025

Related U.S. Application Data
(60) Provisional application No. 63/723,073, filed on Nov. 20, 2024.

(51) Int. Cl. A61K 41/00; C07K 16/28

(52) U.S. Cl. CPC A61K 41/0047; C07K 16/2878

(57) ABSTRACT

Disclosed are methods of treating a subject in need thereof comprising applying an alternating electric field to a target site of the subject in need thereof; and administering one or more CD137 agonists to the subject in need thereof. Disclosed are methods of promoting an effector T (Teff) cell immune response, decreasing immune suppression, decreasing mitochondrial respiration of regulatory T (Treg) cells, and reducing expansion of Treg cells in a subject in need thereof comprising applying an alternating electric field to a target site of the subject in need thereof; and administering one or more CD137 agonists to the subject in need thereof.

### CROSS-REFERENCE TO RELATED APPLICATIONS

This application claims the benefit of U.S. Provisional Ser. No. 63/723,073, filed on Nov. 20, 2024, which is incorporated by reference herein in its entirety.

### BACKGROUND

CD137, also referred to as 4-1BB or TNF receptor superfamily member 9 (TNFRSF9), is a highly potent costimulatory molecule expressed in T and NK cells. CD137 can be expressed as a transmembrane or as a soluble protein. Increased levels of soluble CD137 (sCD137) can be linked to worse outcomes in various diseases by inhibiting the immune system's anti-tumor response and contributing to inflammation.

Therefore, in the presence of increased sCD137, treating with CD137 agonists can be a therapeutic approach to overcome the negative effects of sCD137.

### BRIEF SUMMARY

Disclosed are methods of treating a subject in need thereof comprising applying an alternating electric field to a target site of the subject in need thereof; and administering one or more CD137 agonists to the subject in need thereof.

Disclosed are methods of promoting an effector T (Teff) cell immune response in a subject in need thereof comprising applying an alternating electric field, at a frequency for a period of time, to a target site of the subject in need thereof; and administering one or more CD137 agonists to the subject.

Disclosed are methods of decreasing immune suppression in a subject in need thereof comprising applying an alternating electric field, at a frequency for a period of time, to a target site of the subject in need thereof; and administering one or more CD137 agonists to the subject.

Disclosed are methods of decreasing mitochondrial respiration of regulatory T (Treg) cells in a subject in need thereof comprising applying an alternating electric field, at a frequency for a period of time, to a target site of the subject in need thereof; and administering one or more CD137 agonists to the subject.

Disclosed are methods of reducing expansion of regulatory T (Treg) cells in a subject in need thereof comprising applying an alternating electric field, at a frequency for a period of time, to a target site of the subject in need thereof; and administering one or more CD137 agonists to the subject.

Disclosed are methods of increasing susceptibility of Treg cells to an alternating electric field comprising applying an alternating electric field, at a frequency for a period of time, to a target site of the subject in need thereof; and administering one or more CD137 agonists to the subject.

Disclosed are methods of neutralizing soluble CD137 (sCD137) in a subject comprising applying an alternating electric field, at a frequency for a period of time, to a target site of the subject in need thereof; and administering one or more CD137 agonists to the subject, wherein the alternating electric fields increase sCD137 in the subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an increase in sCD137 between TTField treated cells and control (no TTField) cells. Media from SVZ cell culture (GBM4 and GBM7, n=2) with TTFields (200 kHz, 48 hours, light color bars) compared to control (black bars) were analyzed using Isolight. Data are presented as relative fluorescence units (RFU).

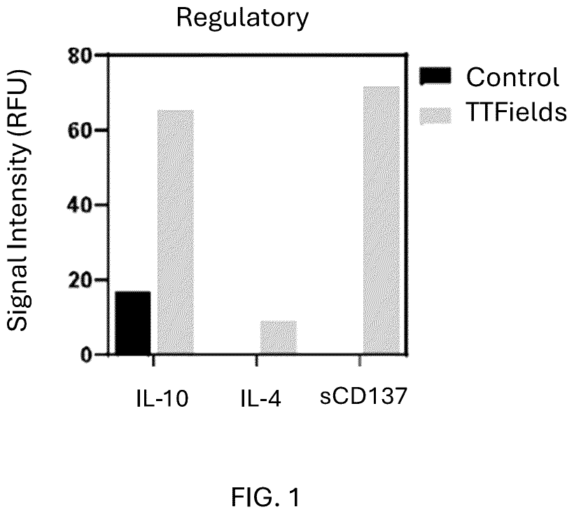

FIG. 2 shows TTFields do not impair T cell viability (T regulatory (Treg) or T conventional (Tconv)). Flow cytometer (FACS) viability staining was performed on T cells and read in a flow cytometer to calculate viable cells.

FIG. 3 shows TTFields selectively reduce Treg expansion.

FIG. 4 shows TTFields have no impact on Tconv cell (T conventional cell) function.

FIG. 5 shows the impact of TTFields (TTF) on Treg cell phenotype.

FIG. 6 shows the mechanisms of action of TTFields treatment on Treg cells phenotype.

FIG. 7 shows the impact of 41BB (CD137) reagents on TTFields (TTF) treated Treg cells.

FIG. 8 shows the impact of 41BB (CD137) reagents on TTFields (TTF) treated Treg cells.

FIG. 9 shows the impact of TTFields (TTF) treatment on Treg cell metabolism.

FIG. 10 shows the impact of TTFields (TTF) treatment in combination with 41BB (CD137) reagents on Treg cell metabolism.

FIG. 11 shows the impact of TTFields (TTF) treatment in combination with 41BB (CD137) reagents on Treg cell metabolism.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cholesterol lowering drug” includes a plurality of such cholesterol lowering drugs, reference to “the cholesterol lowering drug” is a reference to one or more cholesterol lowering drugs and equivalents thereof known to those skilled in the art, and so forth.

The word “or” as used herein means any one member of a particular list and also includes any combination of members of that list.

A “CD137 agonistic antibody” refers to an antibody, preferably a monoclonal antibody, that activates the CD137 (4-1BB) co-stimulatory receptor on immune cells, particularly T cells and natural killer (NK) cells.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the lungs or liver. In some aspects, a cell or population of cells that can be a target site or a target cell include, but are not limited to, a cancer cell (e.g., a brain cancer cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g., a target site such as a cell). In some aspects, the alternating electrical field can be in a single direction or multiple directional. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with cancer.

By “treat” is meant to administer or apply a therapeutic, such as alternating electric fields and a vector, to a subject, such as a human or other mammal (for example, an animal model), that has cancer or has an increased susceptibility for developing cancer, in order to prevent or delay a worsening of the effects of the disease or infection, or to partially or fully reverse the effects of cancer. For example, treating a subject having glioblastoma can comprise delivering a therapeutic to a cell in the subject.

By “prevent” is meant to minimize or decrease the chance that a subject develops cancer.

As used herein, the terms “administering” and “administration” refer to any method of providing a cholesterol lowering drug to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat cancer. In further various aspects, a preparation can be administered prophylactically; that is, administered for prevention of cancer. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, alternating electric field has a frequency. In some aspects, the frequency of the alternating electric field is between 100 and 500 kHz. In some aspects, the frequency of the alternating electric field is between 50 kHz and 1 MHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, alternating electric field has a field strength. In some aspects, the field strength of the alternating electric fields can be between 0.5 and 4 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### C. Methods

CD137 is a co-stimulatory receptor present on T cells and interacts with its ligand, CD137L which is present on antigen present cells, in order to cause signaling through CD137 and aid in activation of the T cell. In some aspects, the binding of a CD137 agonist, such as a CD137 agonistic antibody, to CD137 enhances the immune response to fight cancer and can also be used to suppress autoimmune diseases. For example, a CD137 agonistic antibody can bind to CD137, triggering downstream signaling that leads to increased T cell proliferation, survival, and anti-tumor activity, while also suppressing the activity of regulatory T cells.

In some aspects, alternating electric fields increase soluble CD137 (sCD137). In some aspects, the increase in sCD137 can be due to increased shedding or cleavage of CD137 bound to the cell membrane of T cells. In some aspects, the presence of sCD137 can alter the activation of T cells through membrane bound CD137. In some aspects, CD137L can bind to sCD137 instead of membrane bound CD137, preventing CD137 signaling, and therefore altering or disrupting activation of T cells.

Thus, in some aspects, triggering CD137 signaling, even in the presence of sCD137, can be an effective treatment in subjects with altered CD137 signaling, such as those subjects exposed to an alternating electric field.

### 1. Methods of Treating

Disclosed are methods of treating a subject in need thereof comprising applying an alternating electric field to a target site of the subject in need thereof; and administering one or more CD137 agonists to the subject in need thereof.

In some aspects, the alternating electric field increases the level of sCD137 in the subject. In some aspects, the increase of sCD137 is present in the serum, blood, or biopsy of the subject. In some aspects, the increase in the level of sCD137 is compared to sCD137 levels in the subject prior to applying the alternating electric field. Thus, in some aspects, the methods can further comprise a step of detecting the level of sCD137 in the subject prior to applying the alternating electric field. In some aspects, the increase in the level of sCD137 is compared to a sCD137 standard. In some aspects, a standard is the normal level found in a subject (e.g., healthy subject or cancer patient) not receiving an alternating electric field. For example, in some aspects, a normal level of sCD137 can be below 0.1ng/ml in serum of healthy subject. In some aspects, normal levels of sCD137 can range from 2.47 to 3431.59 pg/ml (mean=121.85 pg/ml, median=17.80 pg/ml) and 4.91 to 270.58 pg/ml (mean=16.37 pg/ml, median=9.974 pg/ml) in cancer patients and healthy donors, respectively.

The disclosed methods can further comprise detecting the presence of the increased level of sCD137 in the subject after applying alternating electric fields and prior to administering one or more CD137 agonists to the subject. Thus, disclosed methods of treating a subject in need thereof comprising applying an alternating electric field to a target site of the subject in need thereof; detecting the presence of increased level of sCD137 in the subject after applying the alternating electric field; and administering one or more CD137 agonists to the subject in need thereof.

The disclosed methods can further comprise detecting the level of sCD137 in the subject prior to applying the alternating electric field to the subject. Thus, disclosed methods of treating a subject in need thereof comprising detecting the presence of a level of sCD137 in the subject prior to applying an alternating electric field; applying an alternating electric field to a target site of the subject in need thereof; and administering one or more CD137 agonists to the subject in need thereof.

The disclosed methods can further comprise detecting the level of sCD137 in the subject prior to applying the alternating electric field to the subject and after applying the alternating electric field to the subject. Thus, disclosed methods of treating a subject in need thereof comprising detecting the presence of a level of sCD137 in the subject prior to applying an alternating electric field; applying an alternating electric field to a target site of the subject in need thereof; detecting the presence of increased level of sCD137 in the subject after applying the alternating electric field; and administering one or more CD137 agonists to the subject in need thereof.

In some aspects, the alternating electric field decreases the level of membrane bound CD137 (mbCD137) in the subject. In some aspects, there is an inverse correlation between the levels of sCD137 and the levels of mbCD137. For example, as mbCD137 levels decrease, sCD137 levels can increase. In some aspects, this inverse correlation can be due to increased shedding or cleavage of CD137 bound

In some aspects, the decrease in the level of mbCD137 is compared to mbCD137 levels in the subject prior to applying the alternating electric field. Thus, in some aspects, the methods can further comprise a step of detecting the level of mbCD137 in the subject prior to applying the alternating electric field. In some aspects, the decrease in the level of mbCD137 is compared to a mbCD137 standard. In some aspects, a standard is the normal level found in a subject (e.g., healthy subject or cancer patient) not receiving an alternating electric field.

The disclosed methods can further comprise detecting the presence of the decreased level of mbCD137 in the subject after applying alternating electric fields and prior to administering one or more CD137 agonists to the subject. Thus, disclosed methods of treating a subject in need thereof comprising applying an alternating electric field to a target site of the subject in need thereof; detecting the presence of decreased level of mbCD137 in the subject after applying the alternating electric field; and administering one or more CD137 agonists to the subject in need thereof.

The disclosed methods can further comprise detecting the level of mbCD137 in the subject prior to applying the alternating electric field to the subject. Thus, disclosed methods of treating a subject in need thereof comprising detecting the presence of a level of mbCD137 in the subject prior to applying an alternating electric field; applying an alternating electric field to a target site of the subject in need thereof; and administering one or more CD137 agonists to the subject in need thereof.

The disclosed methods can further comprise detecting the level of mbCD137 in the subject prior to applying the alternating electric field to the subject and after applying the alternating electric field to the subject. Thus, disclosed methods of treating a subject in need thereof comprising detecting the presence of a level of mbCD137 in the subject prior to applying an alternating electric field; applying an alternating electric field to a target site of the subject in need thereof; detecting the presence of decreased level of mbCD137 in the subject after applying the alternating electric field; and administering one or more CD137 agonists to the subject in need thereof.

In some aspects, the alternating electric field decreases regulatory T cell (Treg) expansion in the subject. In some aspects, the increased sCD137 triggered by the alternating electric field increases Treg cell expansion and reduces activation of T cells that trigger immune response towards the tumor (i.e. Teff cells).

In some aspects, the application of the alternating electrical field and administration of the one or more CD137 agonists promotes an effector T (Teff) cell immune response in the subject. In some aspects, there can be an increase in sCD137 from the alternating electrical field which reduces Teff cells and therefore, the administration of one or more CD137 agonists which can neutralize the sCD137 effects and can promote Teff cells.

In some aspects, the application of the alternating electrical field and administration of the one or more CD137 agonists decreases mitochondrial respiration of regulatory T (Treg) cells in the subject. In some aspects, the decreased mitochondrial respiration of Treg cells can lead to a decrease in Treg cell expansion and/or an increase in Teff cell activation.

In some aspects, the application of the alternating electrical field and administration of the one or more CD137 agonists reduces expansion of Treg cells in the subject. In some aspects, a reduced Treg cell expansion can help promote an increase in Teff cell activation.

In some aspects, the application of the alternating electrical field and administration of the one or more CD137 agonists neutralizes sCD137. In some aspects, the one or more CD137 agonists bind to CD137 since the CD137L can be blocked from binding to the CD137 by the sCD137, thus, the one or more CD137 agonists can neutralize the effects of sCD137 by directly activating membrane bound CD137 on T cells even when CD137L is not available.

In some aspects, the one or more CD137 agonists is a CD137 agonistic antibody. In some aspects, the one or more CD137 agonistic antibody is one or more of Utomilumab (PF-05082566), ATOR 1017, AGEN2373, EU101, CTX-471, ADG106, GEN1046 (DuoBody-PD-L1×4-1BB), PRS-343 (HER2×4-1BB)/Cinrebafusp alfa, RO7122290, ADG206, LVGN6051, ABL503, or any combination thereof.

In some aspects, the one or more CD137 agonists can be a monoclonal antibody, a bispecific antibody, a ligand based fusion protein and/or a cell engager. For example, a monoclonal antibody can be, but is not limited to, Urelumab (BMS-663513), Utomilumab (PF-05082566); a bispecific antibody can be, but is not limited to, PRS-343, GEN1046 and REGN7075; a ligand-based fusion protein can be, but is not limited to, CD137L-Fc fusion and STA551; a cell engager can be, but is not limited to a 4-1BB CAR-T cell. In some aspects, the one or more CD137 agonists can be any molecule that binds and activates CD137.

In some aspects, in a subject in need thereof has cancer. In some aspects, the cancer can be, but is not limited to, brain cancer (e.g. glioblastoma), lung cancer, breast cancer, cervical cancer, colon cancer, gastric cancer, renal cancer, prostate cancer, thyroid cancer, mesothelioma, pancreatic cancer, non-small cell lung, ovarian cancer, hepatic cancer.

In some aspects, the alternating electric field is applied before, after, or simultaneously with administering the one or more CD137 agonists. In some aspects, applying an alternating electric field occurs 1, 2, 3, 4, 5, 6, or 7 days prior to administering the one or more CD137 agonists. In some aspects, applying an alternating electric field occurs 1, 2, 3, 4, 5, 6, or 7 days after administering the one or more CD137 agonists. In some aspects, applying alternating electric fields occurs 1, 2, 3, or 4 weeks prior to administering the one or more CD137 agonists. In some aspects, applying alternating electric fields occurs 1, 2, 3, or 4 weeks after administering the one or more CD137 agonists. In some aspects, the alternating electric fields and the one or more CD137 agonists are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the one or more CD137 agonists is present in the bloodstream prior to applying the alternating electric fields.

In some aspects, the one or more CD137 agonists is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells, brain cancer (e.g. glioblastoma cancer) cells, mesothelioma cancer cells, pancreatic cancer cells, non-small cell lung cancer cells, ovarian cancer cells, hepatic cancer cells.

In some aspects, the target site can be anywhere on a subject's body including, but not limited to, the head, the neck, the torso, and one or more extremity.

In some aspects, the target site comprises Treg cells.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of 1 V/cm RMS. In some aspects, the alternating electric field has a field strength of any of those described herein.

### 2. Methods of Promoting an Effector T (Teff) Cell Immune Response

Shifting the tumor microenvironment towards an effector-dominated immune response can be effective in fighting cancer cells.

Disclosed are methods of promoting an effector T (Teff) cell immune response in a subject in need thereof comprising applying an alternating electric field, at a frequency for a period of time, to a target site of the subject in need thereof; and administering one or more CD137 agonists to the subject.

In some aspects, the combination of alternating electric field and one or more CD137 agonists decreases regulatory T (Treg) cell function, thereby promoting a Teff cell immune response. In some aspects, the decrease in Treg cell function is compared to Treg cell function in the subject prior to applying the combination of alternating electric field and one or more CD137 agonists.

Thus, in some aspects, the methods can further comprise a step of detecting the level of Treg cell function in the subject prior to applying the combination of alternating electric field and one or more CD137 agonists. In some aspects, the decrease in the level of Treg cell function is compared to a Treg cell function standard. In some aspects, a standard is the normal level found in a subject (e.g., healthy subject or cancer patient) not receiving the combination of alternating electric field and one or more CD137 agonists. In some aspects, Treg cell function can be decreased by a decrease in overall Treg cell numbers.

In some aspects, the alternating electric field increases the level of sCD137 in the subject and therefore, in order to promote a shift in the tumor microenvironment towards an more effector-dominated immune response, a CD137 agonist is administered. In some aspects, the methods further comprise detecting the presence of an increased level of sCD137 in the subject after applying alternating electric fields and prior to administering one or more CD137 agonists to the subject. In some aspects, the increase of sCD137 is present in the serum, blood, or biopsy of the subject. In some aspects, the increase in the level of sCD137 is compared to sCD137 levels in the subject prior to applying the alternating electric field. Thus, in some aspects, the methods further comprise detecting the level of sCD137 in the subject prior to applying the alternating electric field to the subject. In some aspects, the increase in the level of sCD137 is compared to a sCD137 standard. In some aspects, a standard is the normal level found in a subject (e.g., healthy subject or cancer patient) not receiving an alternating electric field. For example, in some aspects, a normal level of sCD137 can be below 0.1 ng/ml in serum of healthy subject. In some aspects, normal levels of sCD137 can range from 2.47 to 3431.59 pg/ml (mean=121.85 pg/ml, median=17.80 pg/ml) and 4.91 to 270.58 pg/ml (mean=16.37 pg/ml, median=9.974 pg/ml) in cancer patients and healthy donors, respectively.

In some aspects, the disclosed methods of promoting a Teff cell immune response further comprises detecting the presence of the Teff cell immune response in the subject after both applying an alternating electric field and administering one or more CD137 agonists to the subject. In some aspects, an increase in the Teff cell immune response is compared to Teff cell immune response levels in the subject prior to applying the alternating electric field. Thus, in some aspects, the methods further comprise detecting the Teff cell immune response in the subject prior to applying the alternating electric field to the subject. In some aspects, the increase in the Teff cell immune response is compared to a Teff cell immune response standard. In some aspects, a standard is the normal level found in a subject (e.g., healthy subject or cancer patient) not receiving an alternating electric field and/or one or more CD137 agonists.

In some aspects, the alternating electric field decreases Treg expansion in the subject. In some aspects, the combination of alternating electric field and one or more CD137 agonists decreases mitochondrial respiration of Treg cells. In some aspects, a decrease in Treg expansion and/or a decrease in mitochondrial respiration of Treg cells can slow or decrease the suppression of the immune response. In some aspects, slowing the suppression in the immune response can allow for Teff cells to activate the immune response and ultimately attack cancer cells.

In some aspects, the one or more CD137 agonists is a CD137 agonistic antibody. In some aspects, the one or more CD137 agonistic antibody is one or more of Utomilumab (PF-05082566), ATOR 1017, AGEN2373, EU101, CTX-471, ADG106, GEN1046 (DuoBody-PD-L1×4-1BB), PRS-343 (HER2×4-1BB)/Cinrebafusp alfa, RO7122290, ADG206, LVGN6051, ABL503, or any combination thereof.

In some aspects, the one or more CD137 agonists can be a monoclonal antibody, a bispecific antibody, a ligand based fusion protein and/or a cell engager. For example, a monoclonal antibody can be, but is not limited to, Urelumab (BMS-663513), Utomilumab (PF-05082566); a bispecific antibody can be, but is not limited to, PRS-343, GEN1046 and REGN7075; a ligand-based fusion protein can be, but is not limited to, CD137L-Fc fusion and STA551; a cell engager can be, but is not limited to a 4-1BB CAR-T cell. In some aspects, the one or more CD137 agonists can be any molecule that binds and activates CD137.

In some aspects, in a subject in need thereof has cancer. In some aspects, the cancer can be, but is not limited to, brain cancer (e.g. glioblastoma), lung cancer, breast cancer, cervical cancer, colon cancer, gastric cancer, renal cancer, prostate cancer, thyroid cancer, mesothelioma, pancreatic cancer, non-small cell lung, ovarian cancer, hepatic cancer.

In some aspects, the alternating electric field is applied before, after, or simultaneously with administering the one or more CD137 agonists. In some aspects, applying an alternating electric field occurs 1, 2, 3, 4, 5, 6, or 7 days prior to administering the one or more CD137 agonists. In some aspects, applying an alternating electric field occurs 1, 2, 3, 4, 5, 6, or 7 days after administering the one or more CD137 agonists. In some aspects, applying alternating electric fields occurs 1, 2, 3, or 4 weeks prior to administering the one or more CD137 agonists. In some aspects, applying alternating electric fields occurs 1, 2, 3, or 4 weeks after administering the one or more CD137 agonists. In some aspects, the alternating electric fields and the one or more CD137 agonists are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the one or more CD137 agonists is present in the bloodstream prior to applying the alternating electric fields.

In some aspects, the one or more CD137 agonists is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells, brain cancer (e.g. glioblastoma cancer) cells, mesothelioma cancer cells, pancreatic cancer cells, non-small cell lung cancer cells, ovarian cancer cells, hepatic cancer cells.

In some aspects, the target site can be anywhere on a subject's body including, but not limited to, the head, the neck, the torso, and one or more extremity.

In some aspects, the target site comprises Treg cells.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of 1 V/cm RMS. In some aspects, the alternating electric field has a field strength of any of those described herein.

### 3. Methods of Decreasing Immune Suppression

Disclosed are methods of decreasing immune suppression in a subject in need thereof comprising applying an alternating electric field, at a frequency for a period of time, to a target site of the subject in need thereof; and administering one or more CD137 agonists to the subject.

In some aspects, measuring decreasing immune suppression can be performed by examining quantity of suppressive immune cell population by flow cytometry (FACS) or immunohistological staining (IHC) of the tumor and identifying a decrease in Tregs, myeloid-derived suppressor cells or tumor associated macrophages. In some aspects, an increase in M1 macrophages can indicate a less suppressed immune response and more pro-tumor immunity. In some aspects, measuring decreasing immune suppression can be performed by examining a cytokine profile for serum samples and evaluate for either a reduction in IL-10 and TGF beta, and/or an increase in interferon gamma, IL-2 or TNF alpha. In some aspects, measuring decreasing immune suppression can be performed by an assessment of immune checkpoint molecule expression either with FACS or IHC-a reduction in expression of PD-1, PD-L1, CTLA-4, LAG-3 or TIM-3 on T-cells. In some aspects, measuring decreasing immune suppression can be evaluated by performing functionality tests on T-cells (derived from blood or tumor samples) which can be evaluated for secretion of proinflammatory markers, proliferation or cytotoxic activity.

In some aspects, the combination of alternating electric field and one or more CD137 agonists decreases regulatory T (Treg) cell function, thereby promoting a Teff cell immune response. In some aspects, the decrease in Treg cell function is compared to Treg cell function in the subject prior to applying the combination of alternating electric field and one or more CD137 agonists.

In some aspects, the methods can further comprise detecting a Treg cell immune response in the subject prior to applying an alternating electric field to the subject. In some aspects, the methods can further comprise a step of detecting the level of Treg cell function in the subject prior to applying the combination of alternating electric field and one or more CD137 agonists. In some aspects, the decrease in the level of Treg cell function is compared to a Treg cell function standard. In some aspects, a standard is the normal level found in a subject (e.g., healthy subject or cancer patient) not receiving the combination of alternating electric field and one or more CD137 agonists. In some aspects, Treg cell function can be decreased by a decrease in overall Treg cell numbers.

In some aspects, the alternating electric field increases the level of sCD137 in the subject and therefore, in order to promote a shift in the tumor microenvironment towards an more effector-dominated immune response, a CD137 agonist is administered. In some aspects, the methods further comprise detecting the presence of an increased level of sCD137 in the subject after applying alternating electric fields and prior to administering one or more CD137 agonists to the subject. In some aspects, the increase of sCD137 is present in the serum, blood, or biopsy of the subject. In some aspects, the increase in the level of sCD137 is compared to sCD137 levels in the subject prior to applying the alternating electric field. Thus, in some aspects, the methods further comprise detecting the level of sCD137 in the subject prior to applying the alternating electric field to the subject. In some aspects, the increase in the level of sCD137 is compared to a sCD137 standard. In some aspects, a standard is the normal level found in a subject (e.g., healthy subject or cancer patient) not receiving an alternating electric field. For example, in some aspects, a normal level of sCD137 can be below 0.1 ng/ml in serum of healthy subject. In some aspects, normal levels of sCD137 can range from 2.47 to 3431.59 pg/ml (mean=121.85 pg/ml, median=17.80 pg/ml) and 4.91 to 270.58 pg/ml (mean=16.37 pg/ml, median=9.974 pg/ml) in cancer patients and healthy donors, respectively.

In some aspects, the alternating electric field decreases Treg expansion in the subject. In some aspects, the combination of alternating electric field and one or more CD137 agonists decreases mitochondrial respiration of Treg cells. In some aspects, a decrease in Treg expansion and/or a decrease in mitochondrial respiration of Treg cells can slow or decrease the suppression of the immune response. In some aspects, slowing the suppression in the immune response can allow for Teff cells to activate the immune response and ultimately attack cancer cells.

In some aspects, the one or more CD137 agonists is a CD137 agonistic antibody. In some aspects, the one or more CD137 agonistic antibody is one or more of Utomilumab (PF-05082566), ATOR 1017, AGEN2373, EU101, CTX-471, ADG106, GEN1046 (DuoBody-PD-L1×4-1BB), PRS-343 (HER2×4-1BB)/Cinrebafusp alfa, RO7122290, ADG206, LVGN6051, ABL503, or any combination thereof.

In some aspects, the one or more CD137 agonists can be a monoclonal antibody, a bispecific antibody, a ligand based fusion protein and/or a cell engager. For example, a monoclonal antibody can be, but is not limited to, Urelumab (BMS-663513), Utomilumab (PF-05082566); a bispecific antibody can be, but is not limited to, PRS-343, GEN1046 and REGN7075; a ligand-based fusion protein can be, but is not limited to, CD137L-Fc fusion and STA551; a cell engager can be, but is not limited to a 4-1BB CAR-T cell. In some aspects, the one or more CD137 agonists can be any molecule that binds and activates CD137.

In some aspects, in a subject in need thereof has cancer. In some aspects, the cancer can be, but is not limited to, brain cancer (e.g. glioblastoma), lung cancer, breast cancer, cervical cancer, colon cancer, gastric cancer, renal cancer, prostate cancer, thyroid cancer, mesothelioma, pancreatic cancer, non-small cell lung, ovarian cancer, hepatic cancer.

In some aspects, the alternating electric field is applied before, after, or simultaneously with administering the one or more CD137 agonists. In some aspects, applying an alternating electric field occurs 1, 2, 3, 4, 5, 6, or 7 days prior to administering the one or more CD137 agonists. In some aspects, applying an alternating electric field occurs 1, 2, 3, 4, 5, 6, or 7 days after administering the one or more CD137 agonists. In some aspects, applying alternating electric fields occurs 1, 2, 3, or 4 weeks prior to administering the one or more CD137 agonists. In some aspects, applying alternating electric fields occurs 1, 2, 3, or 4 weeks after administering the one or more CD137 agonists. In some aspects, the alternating electric fields and the one or more CD137 agonists are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the one or more CD137 agonists is present in the bloodstream prior to applying the alternating electric fields.

In some aspects, the one or more CD137 agonists is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells, brain cancer (e.g. glioblastoma cancer) cells, mesothelioma cancer cells, pancreatic cancer cells, non-small cell lung cancer cells, ovarian cancer cells, hepatic cancer cells.

In some aspects, the target site can be anywhere on a subject's body including, but not limited to, the head, the neck, the torso, and one or more extremity.

In some aspects, the target site comprises Treg cells.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of 1 V/cm RMS. In some aspects, the alternating electric field has a field strength of any of those described herein.

### 4. Methods of Decreasing Mitochondrial Respiration

In some aspects, mitochondrial respiration uses nutrients like glucose to produce energy, while cells take up these nutrients from their environment through processes like glucose uptake and transport across cell membranes. In some aspects, Treg cells can take up nutrients so that T cells that are anti-tumorigenic (such as Teff cells) are not able to get sufficient nutrients. Thus, in some aspects, switching this metabolic response from Treg cells having increased metabolism to Teff cells having increased metabolism so that Teff cells get nutrients and are able to kill tumor cells is beneficial to the microenvironment.

In some aspects, an alternating electric field decreases mitochondrial respiration of Treg cells

Disclosed are methods of decreasing mitochondrial respiration of regulatory T (Treg) cells in a subject in need thereof comprising applying an alternating electric field, at a frequency for a period of time, to a target site of the subject in need thereof; and administering one or more CD137 agonists to the subject.

In some aspects, decreasing mitochondrial respiration can be a statistically significant reduction in oxygen-consuming, electron-transport-driven ATP production which can be examined in live T cells compared with a control/baseline. In some aspects, decreasing mitochondrial respiration can be evaluated by Seahorse XF “Mito Stress Test,” high-resolution respirometry (Oroboros O2k/Clark electrode), respiratory chain complex activities (I-IV), and/or cellular ATP production rate partitioning.

In some aspects, the combination of an alternating electric field and one or more CD137 agonists increases a Teff cell immune response. In some aspects, the decrease in mitochondrial respiration of Treg cells causes a decrease in Treg cells which allows for an increase in Teff cells. In some aspects, the combination of the alternating electric field and one or more CD137 agonist decreases mitochondrial respiration of Treg cells, thereby decreasing Treg cell function.

Thus, in some aspects, the methods further comprise detecting Treg cell mitochondrial respiration in the subject after applying alternating electric fields and before administering one or more CD137 agonists to the subject. In some aspects, the methods further comprise detecting a Treg cell mitochondrial respiration in the subject prior to applying an alternating electric field to the subject. In some aspects, the methods further comprise detecting Treg cell mitochondrial respiration in the subject after applying an alternating electric field and administering one or more CD137 agonists to the subject.

In some aspects, the one or more CD137 agonists is a CD137 agonistic antibody. In some aspects, the one or more CD137 agonistic antibody is one or more of Utomilumab (PF-05082566), ATOR 1017, AGEN2373, EU101, CTX-471, ADG106, GEN1046 (DuoBody-PD-L1×4-1BB), PRS-343 (HER2×4-1BB)/Cinrebafusp alfa, RO7122290, ADG206, LVGN6051, ABL503, or any combination thereof.

In some aspects, the one or more CD137 agonists can be a monoclonal antibody, a bispecific antibody, a ligand based fusion protein and/or a cell engager. For example, a monoclonal antibody can be, but is not limited to, Urelumab (BMS-663513), Utomilumab (PF-05082566); a bispecific antibody can be, but is not limited to, PRS-343, GEN1046 and REGN7075; a ligand-based fusion protein can be, but is not limited to, CD137L-Fc fusion and STA551; a cell engager can be, but is not limited to a 4-1BB CAR-T cell. In some aspects, the one or more CD137 agonists can be any molecule that binds and activates CD137.

In some aspects, in a subject in need thereof has cancer. In some aspects, the cancer can be, but is not limited to, brain cancer (e.g. glioblastoma), lung cancer, breast cancer, cervical cancer, colon cancer, gastric cancer, renal cancer, prostate cancer, thyroid cancer, mesothelioma, pancreatic cancer, non-small cell lung, ovarian cancer, hepatic cancer.

In some aspects, the alternating electric field is applied before, after, or simultaneously with administering the one or more CD137 agonists. In some aspects, applying an alternating electric field occurs 1, 2, 3, 4, 5, 6, or 7 days prior to administering the one or more CD137 agonists. In some aspects, applying an alternating electric field occurs 1, 2, 3, 4, 5, 6, or 7 days after administering the one or more CD137 agonists. In some aspects, applying alternating electric fields occurs 1, 2, 3, or 4 weeks prior to administering the one or more CD137 agonists. In some aspects, applying alternating electric fields occurs 1, 2, 3, or 4 weeks after administering the one or more CD137 agonists. In some aspects, the alternating electric fields and the one or more CD137 agonists are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the one or more CD137 agonists is present in the bloodstream prior to applying the alternating electric fields.

In some aspects, the one or more CD137 agonists is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells, brain cancer (e.g. glioblastoma cancer) cells, mesothelioma cancer cells, pancreatic cancer cells, non-small cell lung cancer cells, ovarian cancer cells, hepatic cancer cells.

In some aspects, the target site can be anywhere on a subject's body including, but not limited to, the head, the neck, the torso, and one or more extremity.

In some aspects, the target site comprises Treg cells.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of 1 V/cm RMS. In some aspects, the alternating electric field has a field strength of any of those described herein.

### 5. Methods of Reducing Expansion of Regulatory T (Treg) Cells

Disclosed are methods of reducing expansion of regulatory T (Treg) cells in a subject in need thereof comprising applying an alternating electric field, at a frequency for a period of time, to a target site of the subject in need thereof; and administering one or more CD137 agonists to the subject.

In some aspects, a reduction in expansion of Treg cells can be determined by a reduction in total Treg cell (e.g., CD4+ CD25+ FoxP3+ cell) number. In some aspects, cell number can be counted by flow cytometry.

In some aspects, expansion of conventional T (Tconv) cells is not reduced. In some aspects, Tconv cells are responsible for executing the adaptive immune response, such as attacking infected cells or cancer cells. Thus, not reducing Tconv cell expansion while simultaneously reducing expansion of Treg cells is an effective treatment.

Thus, in some aspects, the methods further comprise detecting expansion of Treg cells in the subject after applying alternating electric fields and before administering one or more CD137 agonists to the subject. In some aspects, the methods further comprise detecting a Treg cell expansion in the subject prior to applying an alternating electric field to the subject. In some aspects, the Treg cell expansion detected prior to applying an alternating electric field can be used as a baseline level to compare the Treg cell expansion after applying alternating electric fields. In some aspects, the methods further comprise detecting Treg cell expansion in the subject after applying an alternating electric field and administering one or more CD137 agonists to the subject. In some aspects, the difference between Treg cell expansion prior to applying an alternating electric field and after applying an alternating electric field and administering one or more CD137 agonists can be used to assess whether there was an increase or decrease in Treg cell expansion.

In some aspects, the combination of an alternating electric field and one or more CD137 agonists decreases mitochondrial respiration of Treg cells, thereby decreasing Treg cell expansion. In some aspects, the decrease in mitochondrial respiration of Treg cells causes a decrease in Treg cells which allows for an increase in Teff cells.

In some aspects, the one or more CD137 agonists is a CD137 agonistic antibody. In some aspects, the one or more CD137 agonistic antibody is one or more of Utomilumab (PF-05082566), ATOR 1017, AGEN2373, EU101, CTX-471, ADG106, GEN1046 (DuoBody-PD-L1×4-1BB), PRS-343 (HER2×4-1BB)/Cinrebafusp alfa, RO7122290, ADG206, LVGN6051, ABL503, or any combination thereof.

In some aspects, the one or more CD137 agonists can be a monoclonal antibody, a bispecific antibody, a ligand based fusion protein and/or a cell engager. For example, a monoclonal antibody can be, but is not limited to, Urelumab (BMS-663513), Utomilumab (PF-05082566); a bispecific antibody can be, but is not limited to, PRS-343, GEN1046 and REGN7075; a ligand-based fusion protein can be, but is not limited to, CD137L-Fc fusion and STA551; a cell engager can be, but is not limited to a 4-1BB CAR-T cell. In some aspects, the one or more CD137 agonists can be any molecule that binds and activates CD137.

In some aspects, in a subject in need thereof has cancer. In some aspects, the cancer can be, but is not limited to, brain cancer (e.g. glioblastoma), lung cancer, breast cancer, cervical cancer, colon cancer, gastric cancer, renal cancer, prostate cancer, thyroid cancer, mesothelioma, pancreatic cancer, non-small cell lung, ovarian cancer, hepatic cancer.

In some aspects, the alternating electric field is applied before, after, or simultaneously with administering the one or more CD137 agonists. In some aspects, applying an alternating electric field occurs 1, 2, 3, 4, 5, 6, or 7 days prior to administering the one or more CD137 agonists. In some aspects, applying an alternating electric field occurs 1, 2, 3, 4, 5, 6, or 7 days after administering the one or more CD137 agonists. In some aspects, applying alternating electric fields occurs 1, 2, 3, or 4 weeks prior to administering the one or more CD137 agonists. In some aspects, applying alternating electric fields occurs 1, 2, 3, or 4 weeks after administering the one or more CD137 agonists. In some aspects, the alternating electric fields and the one or more CD137 agonists are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the one or more CD137 agonists is present in the bloodstream prior to applying the alternating electric fields.

In some aspects, the one or more CD137 agonists is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells, brain cancer (e.g. glioblastoma cancer) cells, mesothelioma cancer cells, pancreatic cancer cells, non-small cell lung cancer cells, ovarian cancer cells, hepatic cancer cells.

In some aspects, the target site can be anywhere on a subject's body including, but not limited to, the head, the neck, the torso, and one or more extremity.

In some aspects, the target site comprises Treg cells.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of 1 V/cm RMS. In some aspects, the alternating electric field has a field strength of any of those described herein.

### 6. Methods of Increasing Susceptibility of Treg Cells to an Alternating Electric Field

Disclosed are methods of increasing susceptibility of Treg cells to an alternating electric field comprising applying an alternating electric field, at a frequency for a period of time, to a target site of the subject in need thereof; and administering one or more CD137 agonists to the subject. Thus, in some aspects, Treg cells are more susceptible to an alternating electric field in the presence of a CD137 agonist. In some aspects, increasing susceptibility of Treg cells to an alternating electric field by administering one or more CD137 agonists can also be referred to as priming Treg cells for alternating electric field treatment by administering one or more CD137 agonists.

In some aspects, increasing susceptibility of the Treg cells to an alternating electric field results in an increased inhibitory effect on the Treg cells. For example, in the presence of an alternating electric field and a CD137 agonist, Treg cells can be decreased or inhibited more than in the presence of an alternating electric field alone.

In some aspects, the combination of an alternating electric field and one or more CD137 agonists decreases Treg expansion.

In some aspects, the combination of an alternating electric field and one or more CD137 agonists increases the alternating electric field ability to decrease Treg expansion.

In some aspects, the one or more CD137 agonists is a CD137 agonistic antibody. In some aspects, the one or more CD137 agonistic antibody is one or more of Utomilumab (PF-05082566), ATOR 1017, AGEN2373, EU101, CTX-471, ADG106, GEN1046 (DuoBody-PD-L1×4-1BB), PRS-343 (HER2×4-1BB)/Cinrebafusp alfa, RO7122290, ADG206, LVGN6051, ABL503, or any combination thereof.

In some aspects, the one or more CD137 agonists can be a monoclonal antibody, a bispecific antibody, a ligand based fusion protein and/or a cell engager. For example, a monoclonal antibody can be, but is not limited to, Urelumab (BMS-663513), Utomilumab (PF-05082566); a bispecific antibody can be, but is not limited to, PRS-343, GEN1046 and REGN7075; a ligand-based fusion protein can be, but is not limited to, CD137L-Fc fusion and STA551; a cell engager can be, but is not limited to a 4-1BB CAR-T cell. In some aspects, the one or more CD137 agonists can be any molecule that binds and activates CD137.

In some aspects, in a subject in need thereof has cancer. In some aspects, the cancer can be, but is not limited to, brain cancer (e.g. glioblastoma), lung cancer, breast cancer, cervical cancer, colon cancer, gastric cancer, renal cancer, prostate cancer, thyroid cancer, mesothelioma, pancreatic cancer, non-small cell lung, ovarian cancer, hepatic cancer.

In some aspects, the alternating electric field is applied before, after, or simultaneously with administering the one or more CD137 agonists. In some aspects, applying an alternating electric field occurs 1, 2, 3, 4, 5, 6, or 7 days prior to administering the one or more CD137 agonists. In some aspects, applying an alternating electric field occurs 1, 2, 3, 4, 5, 6, or 7 days after administering the one or more CD137 agonists. In some aspects, applying alternating electric fields occurs 1, 2, 3, or 4 weeks prior to administering the one or more CD137 agonists. In some aspects, applying alternating electric fields occurs 1, 2, 3, or 4 weeks after administering the one or more CD137 agonists. In some aspects, the alternating electric fields and the one or more CD137 agonists are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the one or more CD137 agonists is present in the bloodstream prior to applying the alternating electric fields.

In some aspects, the one or more CD137 agonists is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells, brain cancer (e.g. glioblastoma cancer) cells, mesothelioma cancer cells, pancreatic cancer cells, non-small cell lung cancer cells, ovarian cancer cells, hepatic cancer cells.

In some aspects, the target site can be anywhere on a subject's body including, but not limited to, the head, the neck, the torso, and one or more extremity.

In some aspects, the target site comprises Treg cells.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of 1 V/cm RMS. In some aspects, the alternating electric field has a field strength of any of those described herein.

### 7. Methods of Neutralizing sCD137

In some aspects, alternating electric fields increase sCD137. In some aspects, the increase in sCD137 can be due to increased shedding or cleavage of CD137 bound to the cell membrane of T cells. In some aspects, the presence of sCD137 can alter the activation of T cells by bind to CD137L, thus preventing CD137L from binding to mbCD137 and activating T cells. Thus, in some aspects, disclosed are methods of neutralizing the effects of sCD137 by directly activating membrane bound CD137 on T cells with a CD137 agonist.

Disclosed are methods of neutralizing soluble CD137 (sCD137) in a subject comprising applying an alternating electric field, at a frequency for a period of time, to a target site of the subject in need thereof; and administering one or more CD137 agonists to the subject, wherein the alternating electric fields increase sCD137 in the subject.

In some aspects, the one or more CD137 agonists can bind to CD137 on the surface of T cells in the subject.

In some aspects, the one or more CD137 agonists enhance T cell activation and/or proliferation. In some aspects, the enhanced T cell activation and/or proliferation is due to stimulation of the CD137 pathway on the T cell.

In some aspects, the methods further comprise detecting the presence of increased sCD137 in the subject after applying alternating electric fields and prior to administering one or more CD137 agonists to the subject. In some aspects, the increase of sCD137 is present in the serum, blood, or biopsy of the subject. In some aspects, the increase in the level of sCD137 is compared to sCD137 levels in the subject prior to applying the alternating electric field. Thus, in some aspects, the methods further comprise detecting the level of sCD137 in the subject prior to applying the alternating electric field to the subject. In some aspects, the increase in the level of sCD137 is compared to a sCD137 standard. In some aspects, a standard is the normal level found in a subject (e.g., healthy subject or cancer patient) not receiving an alternating electric field. For example, in some aspects, a normal level of sCD137 can be below 0.1 ng/ml in serum of healthy subject. In some aspects, normal levels of sCD137 can range from 2.47 to 3431.59 pg/ml (mean=121.85 pg/ml, median=17.80 pg/ml) and 4.91 to 270.58 pg/ml (mean=16.37 pg/ml, median=9.974 pg/ml) in cancer patients and healthy donors, respectively. In some aspects, determining the presence of sCD137 prior to administering the one or more CD137 agonists confirms the need for then administering the one or more CD137 agonists.

In some aspects, neutralizing sCD137 results in promoting Teff cell immune response in the subject. In some aspects, promoting Teff cell immune response can occur by activating CD137 on the surface of the T cells by administering the one or more CD137 agonists.

In some aspects, neutralizing sCD137 results in decreased immune suppression in a subject. In some aspects, the decreased immune suppression is due to the one or more CD137 agonists decreasing Treg cell function.

In some aspects, neutralizing sCD137 results in decreased mitochondrial respiration of Treg cells. In some aspects, neutralizing sCD137 results in reduced expansion of Treg cells.

In some aspects, the one or more CD137 agonists is a CD137 agonistic antibody. In some aspects, the one or more CD137 agonistic antibody is one or more of Utomilumab (PF-05082566), ATOR 1017, AGEN2373, EU101, CTX-471, ADG106, GEN1046 (DuoBody-PD-L1×4-1BB), PRS-343 (HER2×4-1BB)/Cinrebafusp alfa, RO7122290, ADG206, LVGN6051, ABL503, or any combination thereof.

In some aspects, the one or more CD137 agonists can be a monoclonal antibody, a bispecific antibody, a ligand based fusion protein and/or a cell engager. For example, a monoclonal antibody can be, but is not limited to, Urelumab (BMS-663513), Utomilumab (PF-05082566); a bispecific antibody can be, but is not limited to, PRS-343, GEN1046 and REGN7075; a ligand-based fusion protein can be, but is not limited to, CD137L-Fc fusion and STA551; a cell engager can be, but is not limited to a 4-1BB CAR-T cell. In some aspects, the one or more CD137 agonists can be any molecule that binds and activates CD137.

In some aspects, in a subject in need thereof has cancer. In some aspects, the cancer can be, but is not limited to, brain cancer (e.g. glioblastoma), lung cancer, breast cancer, cervical cancer, colon cancer, gastric cancer, renal cancer, prostate cancer, thyroid cancer, mesothelioma, pancreatic cancer, non-small cell lung, ovarian cancer, hepatic cancer.

In some aspects, the alternating electric field is applied before, after, or simultaneously with administering the one or more CD137 agonists. In some aspects, applying an alternating electric field occurs 1, 2, 3, 4, 5, 6, or 7 days prior to administering the one or more CD137 agonists. In some aspects, applying an alternating electric field occurs 1, 2, 3, 4, 5, 6, or 7 days after administering the one or more CD137 agonists. In some aspects, applying alternating electric fields occurs 1, 2, 3, or 4 weeks prior to administering the one or more CD137 agonists. In some aspects, applying alternating electric fields occurs 1, 2, 3, or 4 weeks after administering the one or more CD137 agonists. In some aspects, the alternating electric fields and the one or more CD137 agonists are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the one or more CD137 agonists is present in the bloodstream prior to applying the alternating electric fields.

In some aspects, the one or more CD137 agonists is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells, brain cancer (e.g. glioblastoma cancer) cells, mesothelioma cancer cells, pancreatic cancer cells, non-small cell lung cancer cells, ovarian cancer cells, hepatic cancer cells.

In some aspects, the target site can be anywhere on a subject's body including, but not limited to, the head, the neck, the torso, and one or more extremity.

In some aspects, the target site comprises Treg cells.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of 1 V/cm RMS. In some aspects, the alternating electric field has a field strength of any of those described herein.

### EXAMPLES

### A. Example 1

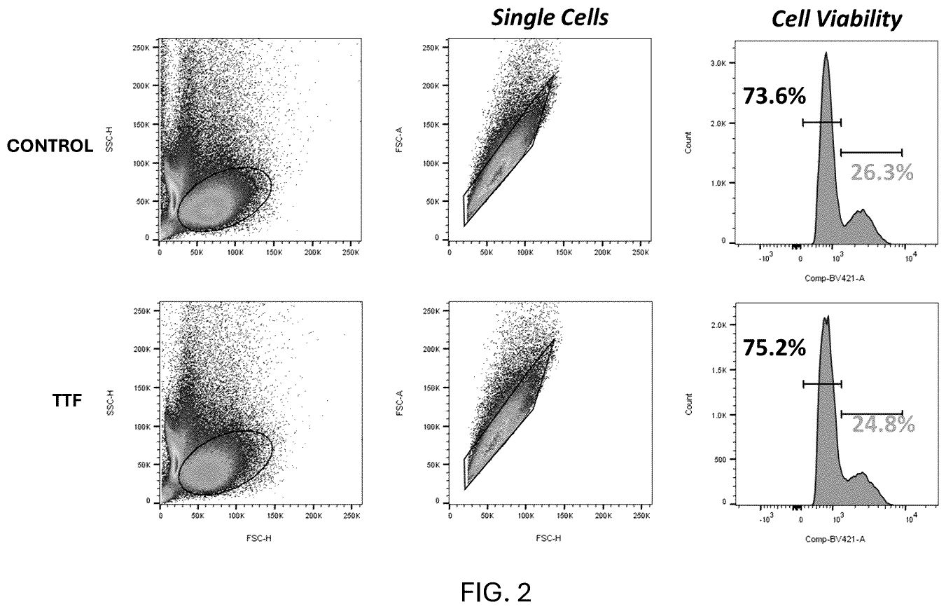

Exposing primary human T cells to TTFields at 150 kHz for 3-5 days does not reduce viability in either regulatory T cells (Tregs) or conventional effector T cells (Tconv) (FIG. 2).

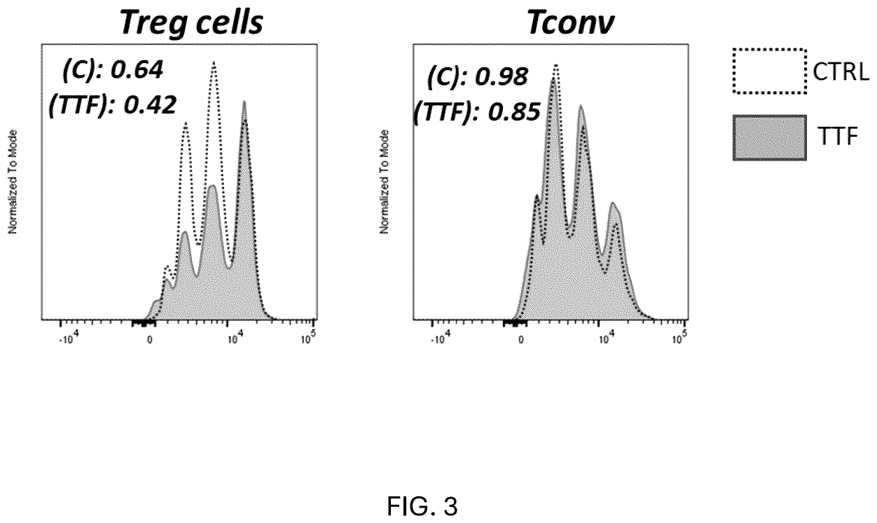

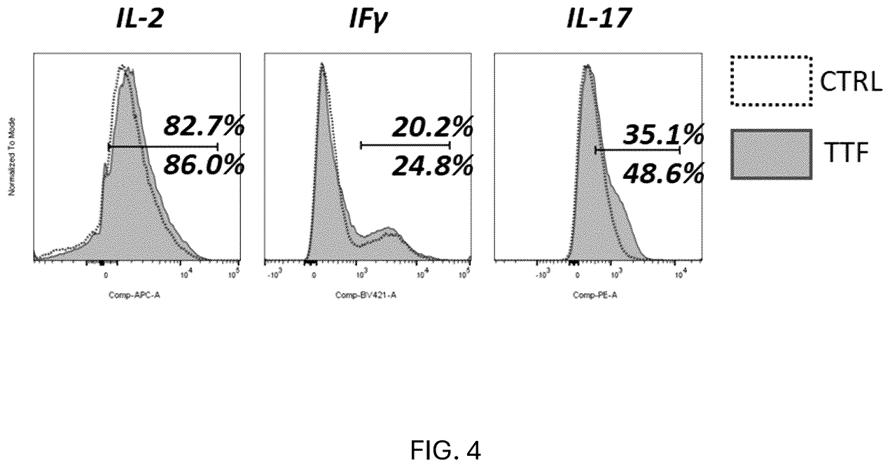

Expansion pattern of T cells was evaluated by FACS. TTFields significantly inhibited Treg proliferation (FIG. 3) while Tconv proliferation and effector function was unchanged (consistent with numbers shown on the slide and the summary statements). FIG. 4 shows that no significant changes were observed in expression of IL-2, interferon gamma (IFgamma) or IL-17 in Tconv cells following TTFields-indicating no change in their functionality.

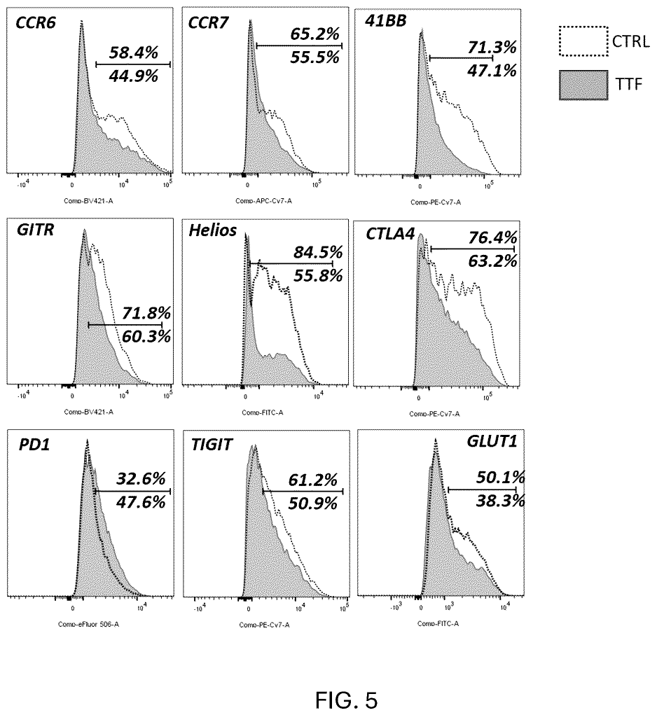

FIG. 5 shows that TTFields remodel the Treg phenotype. Tregs show reduced activation/checkpoint markers including 4-1BB (CD137). FACS analysis of Tregs show that there is a decrease in 4-1BB (CD137), CTLA-4 and Helios expression on Tregs after TTFields application. This data establishes that TTFields application downregulates immunosuppressive, activated Treg profiles.

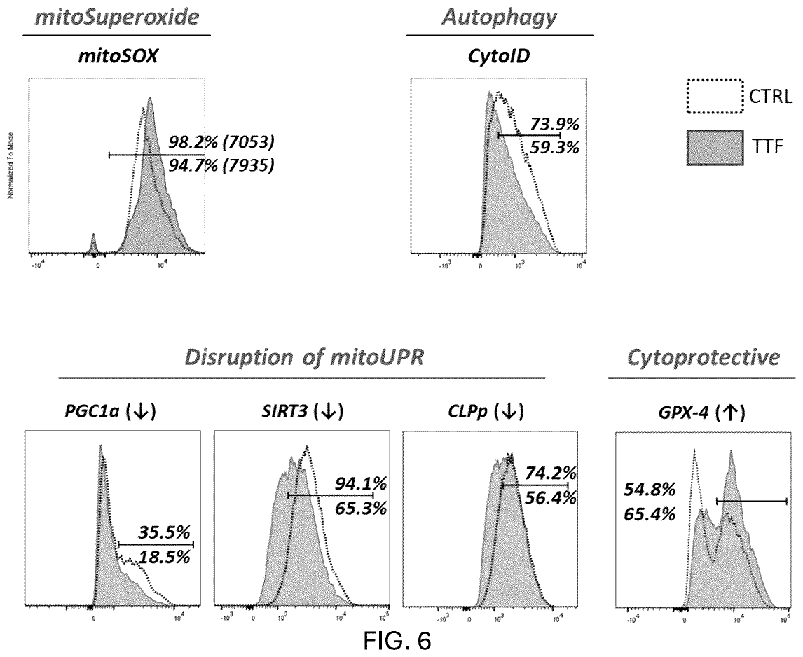

FIG. 6 shows that following TTFields application in Treg cells there is no change in mitochondrial superoxide generation (top left) an increase in autophagy (top right) in addition there was a reduction in disruption of mitoUPR (mitochondrial unfolded protein response, bottom left panels) and a decrease on cytoprotective response (bottom right). Indicative of reduced activity of Treg cells following TTFields treatment.

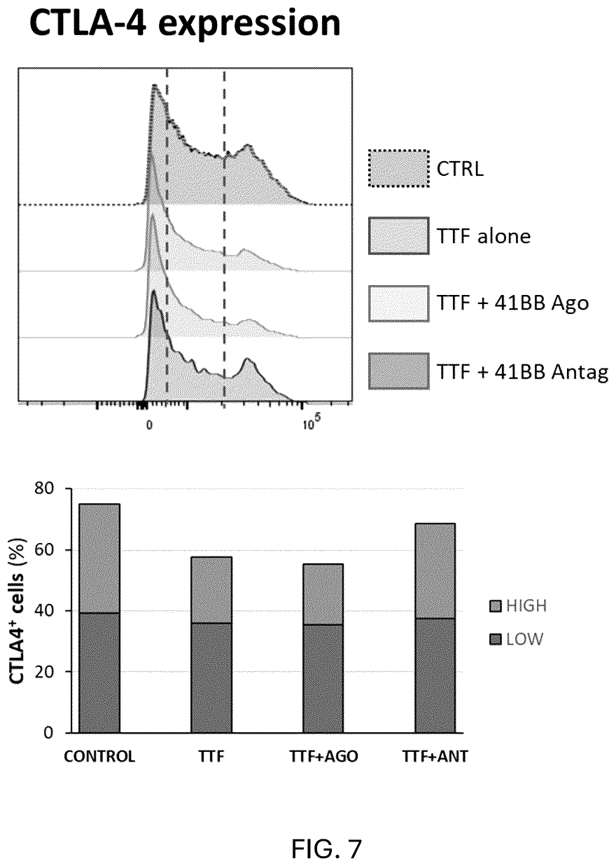

The data represents a comparison in CTLA-4 Tregs expression (FIG. 7) following either untreated, TTFields application, 4-1BB (CD137) agonist or 4-1BB (CD137) antagonist during TTFields exposure. The results show that Tregs become more activated the presence of a 4-1BB (CD137) antagonist and are suppressed following TTFields application concomitant with 4-1BB (CD137) agonist. Thus, agonism of CD137 enhances the TTFields effect on suppression of Tregs.

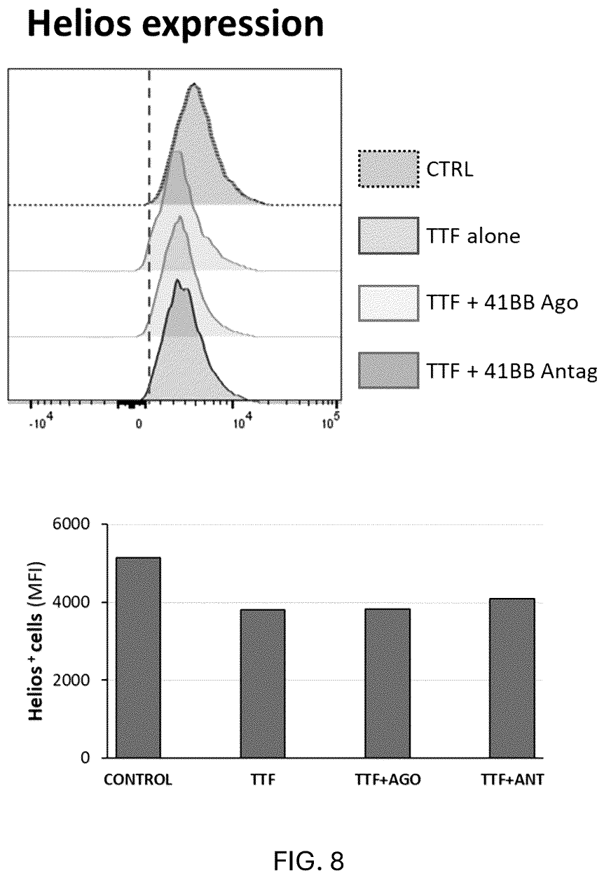

FIG. 8 shows data that represents a comparison in Helios Tregs expression (FIG. 7) following either untreated, TTFields application, 4-1BB (CD137) agonist or 4-1BB (CD137) antagonist during TTFields exposure. The results show that Tregs become more activated the presence of a 4-1BB (CD137) antagonist and are suppressed following TTFields application concomitant with 4-1BB (CD137) agonist. Helios helps maintain FOXP3 expression and Treg identity by repressing genes associated with effector T-cell function.

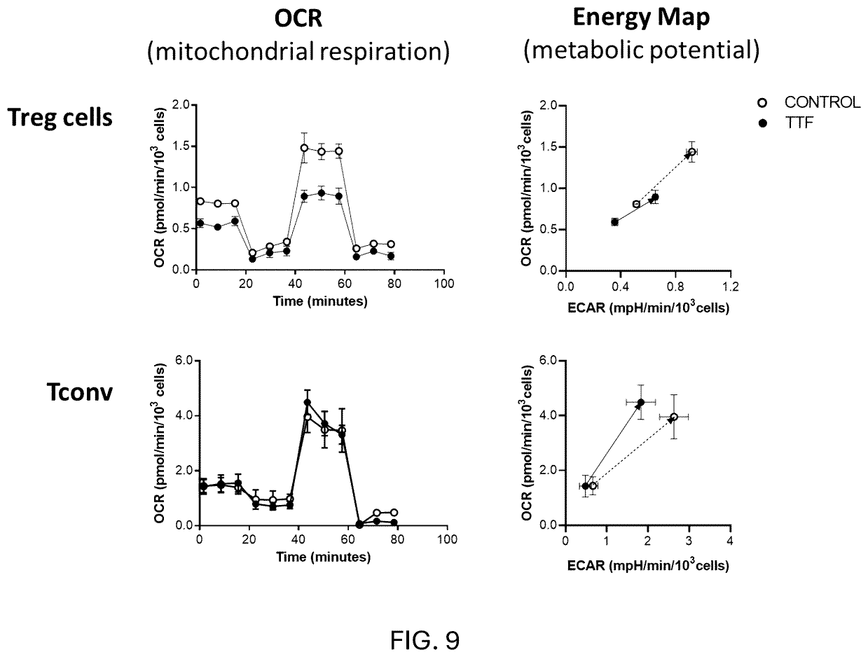

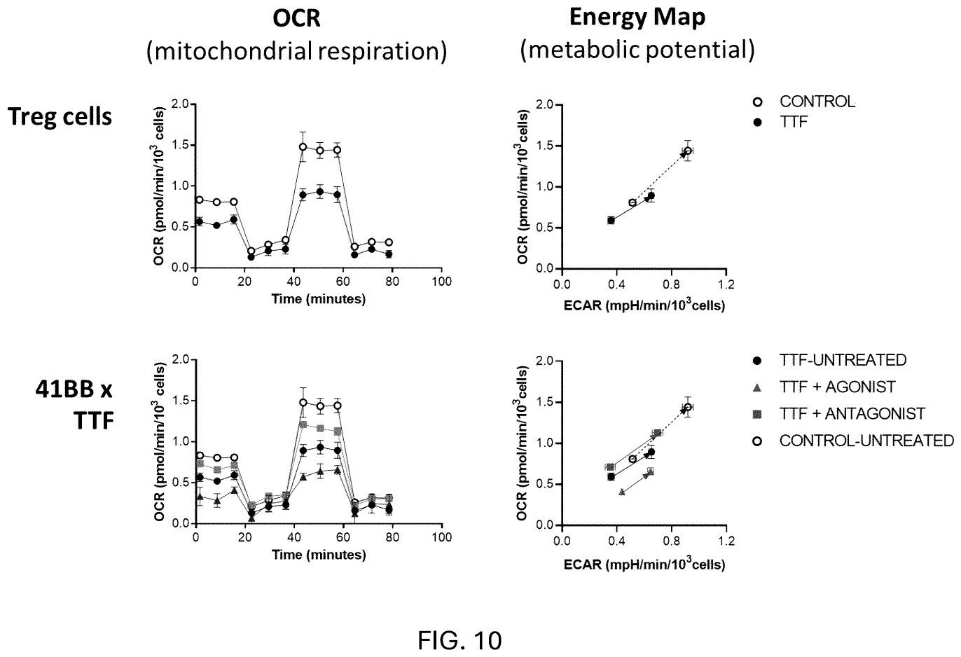

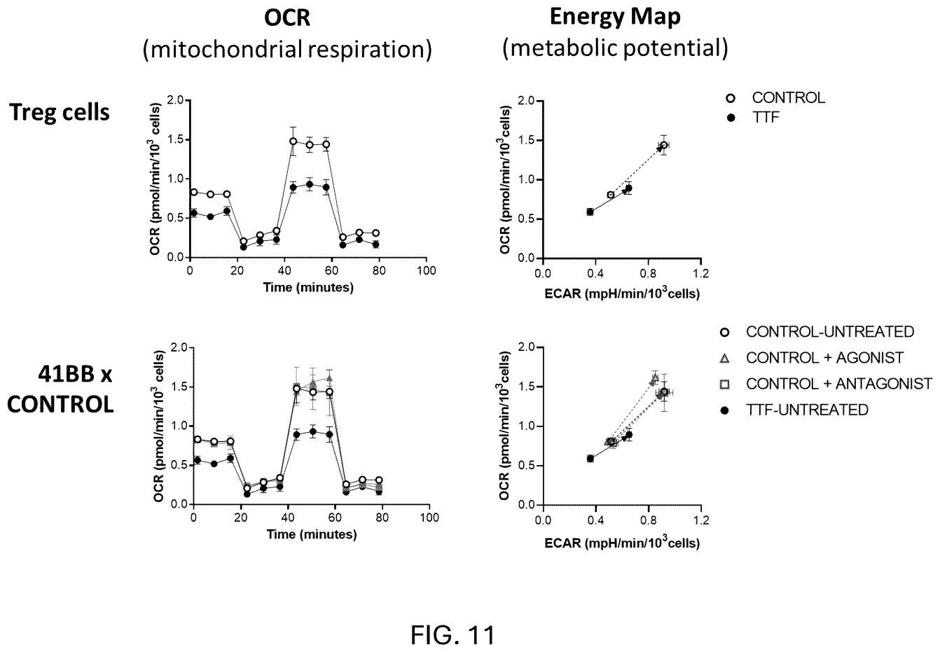

FIGS. 9-11 show that TTFields induce Treg-specific mitochondrial stress and metabolic dysfunction (not seen in Tconv). FIG. 9-11 indicate in various comparisons the mitochondrial metabolic activity which is indicative of T cell activation in Tregs after TTFields. There is a reduction in mitochondrial metabolism with no comparable disruption in Tconv cells. Furthermore, when TTFields are concomitant with 4-1BB (CD137) agonist this reduction in Treg activity is enhanced representing less immune suppression that leads to activation of Tconv cells to target the tumor. While TTFields concomitantly with 4-1BB (CD137) antagonist show more activation of Tregs and less immune suppression to TTFields application alone.

### SUMMARY

TTF treatment at 150 kHz for 3-5 days does not affect Treg or Tconv cell viability. TTF treatment can significantly reduce the expansion of Tregs, but not of Tconv. TTF treatment does not inhibit effector T cell function. TTF treatment triggers mitochondrial oxidative stress, leading to the disruption of mitoUPR and alteration of autophagic responses in Treg cells.

TTF treatment promotes significant phenotypic changes in Treg cells, including reduced expression of the early activation marker 41BB, the immune checkpoint inhibitor CTLA-4, and the Treg maturation marker Helios.

TTF treatment disrupts mitochondrial oxidative metabolism in Treg cells but not in Tconv. TTF-induced mitochondrial metabolic damage associates with the integrity of the 41BB signaling pathway: Treg cells are more susceptible to TTF in the presence of a 41BB (CD137) agonist and exhibit greater resistance to TTF when 41BB (CD137) signaling is blocked.

These findings indicate that the combination of TTF with a 41BB (CD137) agonist may promote a shift in the tumor microenvironment towards an more effector-dominated immune response.

### Embodiments

Embodiment 1: A method of treating a subject in need thereof comprising applying an alternating electric field, at a frequency for a period of time, to a target site of the subject in need thereof; and administering one or more CD137 agonists to the subject in need thereof.
Embodiment 2: The method of embodiment 1, wherein the alternating electric field increases the level of soluble CD137 (sCD137) in the subject.
Embodiment 3: The method of embodiment 2, wherein the increase in the level of sCD137 is compared to a sCD137 standard.
Embodiment 4: The method of embodiment 3, wherein the increase in the level of sCD137 is compared to sCD137 levels in the subject prior to applying the alternating electric field.
Embodiment 5: The method of any of the preceding embodiments, further comprising detecting the presence of the increased level of sCD137 in the subject after applying alternating electric fields and prior to administering one or more CD137 agonists to the subject.
Embodiment 6: The method of any of the preceding embodiments, further comprising detecting the level of sCD137 in the subject prior to applying the alternating electric field to the subject.
Embodiment 7: The method of embodiment 1, wherein the alternating electric field decreases the level of membrane bound CD137 (mbCD137) in the subject.
Embodiment 8: The method of embodiment 8, wherein the decrease in the level of mbCD137 is compared to a mbCD137 standard.
Embodiment 9: The method of any of embodiments 8-9, wherein the decrease in the level of mbCD137 is compared to mbCD137 levels prior to applying the alternating electric field.
Embodiment 10: The method of any of the preceding embodiments, further comprising detecting the presence of the decreased level of mbCD137 in the subject after applying alternating electric fields and prior to administering one or more CD137 to the subject.
Embodiment 11: The method of any of the preceding embodiments, further comprising detecting the level of mbCD137 in the subject prior to applying the alternating electric field to the subject.
Embodiment 12: The method of any of the preceding embodiments, wherein the alternating electric field decreases regulatory T cell (Treg) expansion in the subject.
Embodiment 13: The method of any of the preceding embodiments, wherein the application of the alternating electrical field and administration of the one or more CD137 agonists promotes an effector T (Teff) cell immune response in the subject.
Embodiment 14: The method of any of the preceding embodiments, wherein the application of the alternating electrical field and administration of the one or more CD137 agonists decreases mitochondrial respiration of regulatory T (Treg) cells in the subject.
Embodiment 15: The method of any of the preceding embodiments, wherein the application of the alternating electrical field and administration of the one or more CD137 agonists reduces expansion of Treg cells in the subject.
Embodiment 16: The method of any of the preceding embodiments, wherein the application of the alternating electrical field and administration of the one or more CD137 agonists neutralizes sCD137.
Embodiment 17: A method of promoting an effector T (Teff) cell immune response in a subject comprising applying an alternating electric field, at a frequency for a period of time, to a target site of the subject in need thereof; and administering one or more CD137 agonists to the subject.
Embodiment 18: The method of embodiment 17, wherein the combination of alternating electric field and one or more CD137 agonists decreases regulatory T (Treg) cell function, thereby promoting a Teff cell immune response.
Embodiment 19: The method of any of embodiments 17-18, further comprising detecting the presence of the increased level of sCD137 in the subject after applying alternating electric fields and prior to administering one or more CD137 agonists to the subject.
Embodiment 20: The method of any of embodiments 17-18, further comprising detecting the level of sCD137 in the subject prior to applying the alternating electric field to the subject.
Embodiment 21: The method of any of embodiments 17-20, further comprising detecting the presence of the Teff cell immune response in the subject after applying an alternating electric field and administering one or more CD137 agonists to the subject.
Embodiment 22: The method of any of embodiments 17-21, wherein the alternating electric field decreases Treg expansion in the subject.
Embodiment 23: The method of any of embodiments 17-22, wherein the combination of alternating electric field and one or more CD137 agonists decreases mitochondrial respiration of Treg cells.
Embodiment 24: A method of decreasing immune suppression in a subject in need thereof comprising applying an alternating electric field, at a frequency for a period of time, to a target site of the subject in need thereof; and administering one or more CD137 agonists to the subject.
Embodiment 25: The method of embodiment 24, wherein the combination of alternating electric field and one or more CD137 agonists decreases regulatory T (Treg) cell function, thereby decreasing immune suppressive effects of Treg cells.
Embodiment 26: The method of any of embodiments 24-25, further comprising detecting the presence of the increased level of sCD137 in the subject after applying alternating electric fields and prior to administering one or more CD137 agonistic antibodies to the subject.
Embodiment 27: The method of any of embodiments 24-26, further comprising detecting a Treg cell immune response in the subject prior to applying an alternating electric field to the subject.
Embodiment 28: The method of any of embodiments 24-26, further comprising detecting a Treg cell immune response in the subject after applying an alternating electric field and administering one or more CD137 agonists to the subject.
Embodiment 29: The method of any of embodiments 24-28, wherein the alternating electric field decreases Treg expansion.
Embodiment 30: The method of any of embodiments 24-29, wherein the combination of alternating electric field and one or more CD137 agonists decreases mitochondrial respiration of Treg cells.
Embodiment 31: A method of decreasing mitochondrial respiration of regulatory T (Treg) cells in a subject in need thereof comprising applying an alternating electric field, at a frequency for a period of time, to a target site of the subject in need thereof; and administering one or more CD137 agonists to the subject.
Embodiment 32: The method of embodiment 31, wherein the combination of alternating electric field and one or more CD137 agonistic antibody increases a Teff cell immune response.
Embodiment 33: The method of any one of embodiments 31-32, wherein the combination of alternating electric field and one or more CD137 agonist decreases mitochondrial respiration of Treg cells, thereby decreasing Treg cell function.
Embodiment 34: The method of any of embodiments 31-33, further comprising detecting Treg cell mitochondrial respiration in the subject after applying alternating electric fields and before administering one or more CD137 agonists to the subject.
Embodiment 35: The method of any of embodiments 31-33, further comprising detecting a Treg cell mitochondrial respiration in the subject prior to applying an alternating electric field to the subject.
Embodiment 36: The method of any of embodiments 31-33, further comprising detecting Treg cell mitochondrial respiration in the subject after applying an alternating electric field and administering one or more CD137 agonists to the subject.
Embodiment 37: A method of reducing expansion of regulatory T (Treg) cells in a subject in need thereof comprising applying an alternating electric field, at a frequency for a period of time, to a target site of the subject in need thereof; and administering one or more CD137 agonists to the subject.
Embodiment 38: The method of embodiment 37, wherein expansion of conventional T (Tconv) cells is not reduced.
Embodiment 39: The method of any of embodiments 37-38, further comprising detecting expansion of Treg cells in the subject after applying alternating electric fields and before administering one or more CD137 agonists to the subject.
Embodiment 40: The method of any of embodiments 37-38, further comprising detecting a Treg cell expansion in the subject prior to applying an alternating electric field to the subject.
Embodiment 41: The method of any of embodiments 37-38, further comprising detecting a Treg cell expansion in the subject after applying an alternating electric field and administering one or more CD137 agonists to the subject.
Embodiment 42: The method of any of embodiments 37-41, wherein the combination of alternating electric field and one or more CD137 agonists decreases mitochondrial respiration of Treg cells, thereby decreasing Treg cell expansion.
Embodiment 43: A method of increasing susceptibility of regulatory T (Treg) cells to an alternating electric field comprising applying an alternating electric field, at a frequency for a period of time, to a target site of the subject in need thereof; and administering one or more CD137 agonists to the subject.
Embodiment 44: The method of embodiment 43, wherein increasing susceptibility results in an increased inhibitory effect on the Treg cells.
Embodiment 45: The method of any one of embodiments 43-44, wherein the combination of an alternating electric field and one or more CD137 agonists decreases Treg expansion.
Embodiment 46: The method of any one of embodiments 43-44, wherein the combination of an alternating electric field and one or more CD137 agonists increases the alternating electric field ability to decrease Treg expansion.
Embodiment 47: A method of neutralizing soluble CD137 (sCD137) in a subject comprising applying an alternating electric field, at a frequency for a period of time, to a target site of the subject in need thereof; and administering one or more CD137 agonists to the subject, wherein the alternating electric fields increase sCD137 in the subject.
Embodiment 48: The method of embodiment 47, wherein the one or more CD137 agonists can bind to CD137 receptor on the surface of T cells in the subject.
Embodiment 49: The method of any one of embodiments 47-48, wherein the one or more CD137 agonists enhances T cell activation and/or proliferation.
Embodiment 50: The method of any of embodiments 47-49, further comprising detecting the presence of increased sCD137 in the subject after applying alternating electric fields and before administering one or more CD137 agonists to the subject.
Embodiment 51: The method of any of embodiments 47-50, wherein neutralizing sCD137 results in promoting effector T (Teff) cell immune response in the subject.
Embodiment 52: The method of any of embodiments 47-51, wherein neutralizing sCD137 results in decreased immune suppression in a subject.
Embodiment 53: The method of any of embodiments 47-52, wherein neutralizing sCD137 results in decreased mitochondrial respiration of regulatory T (Treg) cells.
Embodiment 54: The method of any of embodiments 47-53, wherein neutralizing sCD137 results in reduced expansion of regulatory T (Treg) cells
Embodiment 55: The method of any of embodiments 47-54, wherein the target site comprises one or more cancer cells.
Embodiment 56: The method of embodiment 55, wherein the one or more cancer cells are glioblastoma cells.
Embodiment 57: The method of any of the preceding embodiments, wherein the one or more CD137 agonists is a CD137 agonistic antibody.
Embodiment 58: The method of embodiment 57, wherein the one or more CD137 agonistic antibody is one or more of Utomilumab (PF-05082566), ATOR 1017, AGEN2373, EU101, CTX-471, ADG106, GEN1046 (DuoBody-PD-L1×4-1BB), PRS-343 (HER2×4-1BB)/Cinrebafusp alfa, RO7122290, ADG206, LVGN6051, ABL503 or any combination thereof.
Embodiment 59: The method of any of the preceding embodiments, wherein the subject has cancer.
Embodiment 60: The method of embodiment 59, wherein the cancer is brain cancer (e.g. glioblastoma), lung cancer, breast cancer, cervical cancer, colon cancer, gastric cancer, renal cancer, prostate cancer, thyroid cancer, mesothelioma, pancreatic cancer, non-small cell lung, ovarian cancer, hepatic cancer.
Embodiment 61: The method of any of the preceding embodiments, wherein the alternating electric fields are applied before, after, or simultaneously with administering the one or more CD137 agonists.
Embodiment 62: The method of any of the preceding embodiments, wherein the target site comprises Treg cells.
Embodiment 63: The method of any of the preceding embodiments, wherein the alternating electric field has a frequency of between 50 kHz and 1 MHz.
Embodiment 64: The method of any of the preceding embodiments, wherein the alternating electric field has a frequency of between 150 or 250 kHz.
Embodiment 65: The method of any of the preceding embodiments, wherein the alternating electric field has a field strength of between 0.5 and 4 V/cm RMS.
Embodiment 66: The method of any of the preceding embodiments, wherein the alternating electric field has a field strength of 0.9 V/cm RMS.
Embodiment 67: An alternating electric field for use in a method of treating a subject comprising administering one or more CD137 agonists to the subject in need thereof.
Embodiment 68: Embodiment 67, wherein the alternating electric field increases the level of soluble CD137 (sCD137) in the subject.
Embodiment 69: Embodiment 68, wherein the increase in the level of sCD137 is compared to a sCD137 standard.
Embodiment 70: Embodiment 69, wherein the increase in the level of sCD137 is compared to sCD137 levels prior to applying the alternating electric field.
Embodiment 71: Embodiments 67-70, further comprising detecting the presence of the increased level of sCD137 in the subject after applying alternating electric fields and prior to administering one or more CD137 agonists to the subject.
Embodiment 72: Embodiments 67-71, further comprising detecting the level of sCD137 in the subject prior to applying the alternating electric field to the subject.
Embodiment 73: Embodiment 67, wherein the alternating electric field decreases the level of membrane bound CD137 (mbCD137) in the subject.
Embodiment 74: Embodiment 73, wherein the decrease in the level of mbCD137 is compared to a mbCD137 standard.
Embodiment 75: Embodiments 73-74, wherein the decrease in the level of mbCD137 is compared to mbCD137 levels prior to applying the alternating electric field.
Embodiment 76: Embodiments 67-75, further comprising detecting the presence of the decreased level of mbCD137 in the subject after applying alternating electric fields and prior to administering one or more CD137 to the subject.
Embodiment 77: Embodiments 67-76, further comprising detecting the level of mbCD137 in the subject prior to applying the alternating electric field to the subject.
Embodiment 78: Embodiments 67-77, wherein the alternating electric field decreases regulatory T cell (Treg) expansion in the subject.
Embodiment 79: Embodiments 67-78, wherein the application of the alternating electrical field and administration of the one or more CD137 agonists promotes an effector T (Teff) cell immune response in the subject.
Embodiment 80: Embodiments 67-79, wherein the application of the alternating electrical field and administration of the one or more CD137 agonists decreases mitochondrial respiration of regulatory T (Treg) cells in the subject.
Embodiment 81: Embodiments 67-80, wherein the application of the alternating electrical field and administration of the one or more CD137 agonists reduces expansion of Treg cells in the subject.
Embodiment 82: Embodiments 67-81, wherein the application of the alternating electrical field and administration of the one or more CD137 agonists neutralizes sCD137.
Embodiment 83: An alternating electric field for use in a method of promoting an effector T (Teff) cell immune response in a subject comprising administering one or more CD137 agonists to the subject.
Embodiment 84: Embodiment 83, wherein the combination of alternating electric field and one or more CD137 agonists decreases regulatory T (Treg) cell function, thereby promoting a Teff cell immune response.
Embodiment 85: Embodiments 83-84, further comprising detecting the presence of the increased level of sCD137 in the subject after applying alternating electric fields and prior to administering one or more CD137 agonists to the subject.
Embodiment 86: Embodiments 83-85, further comprising detecting the level of sCD137 in the subject prior to applying the alternating electric field to the subject.
Embodiment 87: Embodiments 83-86, further comprising detecting the presence of the Teff cell immune response in the subject after applying an alternating electric field and administering one or more CD137 agonists to the subject.
Embodiment 88: Embodiments 83-87, wherein the alternating electric field decreases Treg expansion in the subject.
Embodiment 89: Embodiments 83-88, wherein the combination of alternating electric field and one or more CD137 agonists decreases mitochondrial respiration of Treg cells.
Embodiment 90: An alternating electric field for use in a method of decreasing immune suppression in a subject in need thereof comprising administering one or more CD137 agonists to the subject.
Embodiment 91: Embodiment 90, wherein the combination of alternating electric field and one or more CD137 agonists decreases regulatory T (Treg) cell function, thereby decreasing immune suppressive effects of Treg cells.
Embodiment 92: Embodiments 90-91, further comprising detecting the presence of the increased level of sCD137 in the subject after applying alternating electric fields and prior to administering one or more CD137 agonistic antibodies to the subject.
Embodiment 93: Embodiments 90-92, further comprising detecting a Treg cell immune response in the subject prior to applying an alternating electric field to the subject.
Embodiment 94: Embodiments 90-93, further comprising detecting a Treg cell immune response in the subject after applying an alternating electric field and administering one or more CD137 agonists to the subject.
Embodiment 95: Embodiments 90-94, wherein the alternating electric field decreases Treg expansion.
Embodiment 96: Embodiments 90-95, wherein the combination of alternating electric field and one or more CD137 agonists decreases mitochondrial respiration of Treg cells.
Embodiment 97: An alternating electric field for use in a method of decreasing mitochondrial respiration of regulatory T (Treg) cells in a subject in need thereof comprising administering one or more CD137 agonists to the subject.
Embodiment 98: Embodiment 97, wherein the combination of alternating electric field and one or more CD137 agonistic antibody increases a Teff cell immune response.
Embodiment 99: Embodiments 97-98, wherein the combination of alternating electric field and one or more CD137 agonist decreases mitochondrial respiration of Treg cells, thereby decreasing Treg cell function.
Embodiment 100: Embodiments 97-99, further comprising detecting Treg cell mitochondrial respiration in the subject after applying alternating electric fields and before administering one or more CD137 agonists to the subject.
Embodiment 101: Embodiments 97-100, further comprising detecting a Treg cell mitochondrial respiration in the subject prior to applying an alternating electric field to the subject.
Embodiment 102: Embodiments 97-101, further comprising detecting Treg cell mitochondrial respiration in the subject after applying an alternating electric field and administering one or more CD137 agonists to the subject.
Embodiment 103: An alternating electric field for use in a method of reducing expansion of regulatory T (Treg) cells in a subject in need thereof comprising administering one or more CD137 agonists to the subject.
Embodiment 104: Embodiment 103, wherein expansion of conventional T (Tconv) cells is not reduced.
Embodiment 105: Embodiments 103-104 further comprising detecting expansion of Treg cells in the subject after applying alternating electric fields and before administering one or more CD137 agonists to the subject.
Embodiment 106: Embodiments 103-105, further comprising detecting a Treg cell expansion in the subject prior to applying an alternating electric field to the subject.
Embodiment 107: Embodiments 103-106, further comprising detecting a Treg cell expansion in the subject after applying an alternating electric field and administering one or more CD137 agonists to the subject.
Embodiment 108: Embodiments 103-107, wherein the combination of alternating electric field and one or more CD137 agonists decreases mitochondrial respiration of Treg cells, thereby decreasing Treg cell expansion.
Embodiment 109: An alternating electric field for use in a method of increasing susceptibility of regulatory T (Treg) cells to an alternating electric field comprising administering one or more CD137 agonists to the subject.
Embodiment 110: Embodiment 109, wherein increasing susceptibility results in an increased inhibitory effect on the Treg cells.
Embodiment 111: Embodiments 109-110, wherein the combination of an alternating electric field and one or more CD137 agonists decreases Treg expansion.
Embodiment 112: Embodiments 109-111, wherein the combination of an alternating electric field and one or more CD137 agonists increases the alternating electric field ability to decrease Treg expansion.
Embodiment 113: An alternating electric field for use in a method of neutralizing soluble CD137 (sCD137) in a subject comprising administering one or more CD137 agonists to the subject, wherein the alternating electric fields increase sCD137 in the subject.
Embodiment 114: Embodiment 113, wherein the one or more CD137 agonists can bind to CD137 receptor on the surface of T cells in the subject.
Embodiment 115: Embodiments 113-114, wherein the one or more CD137 agonists enhances T cell activation and/or proliferation.
Embodiment 116: Embodiments 113-115, further comprising detecting the presence of increased sCD137 in the subject after applying alternating electric fields and before administering one or more CD137 agonists to the subject.
Embodiment 117: Embodiments 113-116, wherein neutralizing sCD137 results in promoting effector T (Teff) cell immune response in the subject.
Embodiment 118: Embodiments 113-117, wherein neutralizing sCD137 results in decreased immune suppression in a subject.
Embodiment 119: Embodiments 113-118, wherein neutralizing sCD137 results in decreased mitochondrial respiration of regulatory T (Treg) cells.
Embodiment 120: Embodiments 113-119, wherein neutralizing sCD137 results in reduced expansion of regulatory T (Treg) cells
Embodiment 121: Embodiments 113-120, wherein the target site comprises one or more cancer cells.
Embodiment 122: Embodiment 121, wherein the one or more cancer cells are glioblastoma cells.
Embodiment 123: Embodiments 67-123, wherein the one or more CD137 agonists is a CD137 agonistic antibody.
Embodiment 124: Embodiment 123, wherein the one or more CD137 agonistic antibody is one or more of Utomilumab (PF-05082566), ATOR 1017, AGEN2373, EU101, CTX-471, ADG106, GEN1046 (DuoBody-PD-L1×4-1BB), PRS-343 (HER2×4-1BB)/Cinrebafusp alfa, RO7122290, ADG206, LVGN6051, ABL503 or any combination thereof.
Embodiment 125: Embodiments 67-125, wherein the subject has cancer.
Embodiment 126: Embodiment 125, wherein the cancer is brain cancer (e.g. glioblastoma), lung cancer, breast cancer, cervical cancer, colon cancer, gastric cancer, renal cancer, prostate cancer, thyroid cancer, mesothelioma, pancreatic cancer, non-small cell lung, ovarian cancer, hepatic cancer.
Embodiment 127: Embodiments 67-126, wherein the alternating electric fields are applied before, after, or simultaneously with administering the one or more CD137 agonists.
Embodiment 128: Embodiments 67-127, wherein the target site comprises Treg cells.
Embodiment 129: Embodiments 67-128, wherein the alternating electric field has a frequency of between 50 kHz and 1 MHz.
Embodiment 130: Embodiments 67-129, wherein the alternating electric field has a frequency of between 150 or 250 kHz.
Embodiment 131: Embodiments 67-130, herein the alternating electric field has a field strength of between 0.5 and 4 V/cm RMS.
Embodiment 132: Embodiments 67-131, wherein the alternating electric field has a field strength of 0.9 V/cm RMS.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject in need thereof comprising:
a) applying an alternating electric field, at a frequency for a period of time, to a target site of the subject in need thereof; and
b) administering one or more CD137 agonists to the subject in need thereof.

2. The method of claim 1, wherein the alternating electric field increases the level of soluble CD137 (sCD137) in the subject.

3. The method of claim 2, wherein the increase in the level of sCD137 is compared to a sCD137 standard or to sCD137 levels in the subject prior to applying the alternating electric field.

4. The method of claim 3, further comprising detecting the presence of the increased level of sCD137 in the subject after applying alternating electric fields and prior to administering one or more CD137 agonists to the subject.

5. The method of claim 1, further comprising detecting the level of sCD137 in the subject prior to applying the alternating electric field to the subject.

6. The method of claim 1, wherein the alternating electric field decreases the level of membrane bound CD137 (mbCD137) in the subject.

7. The method of claim 6, further comprising detecting the presence of the decreased level of mbCD137 in the subject after applying alternating electric fields and prior to administering one or more CD137 to the subject.

8. The method of claim 1, further comprising detecting the level of mbCD137 in the subject prior to applying the alternating electric field to the subject.

9. The method of claim 1, wherein the alternating electric field decreases regulatory T cell (Treg) expansion in the subject.

10. The method of claim 1, wherein the application of the alternating electrical field and administration of the one or more CD137 agonists promotes an effector T (Teff) cell immune response in the subject.

11. The method of claim 1, wherein the application of the alternating electrical field and administration of the one or more CD137 agonists decreases mitochondrial respiration of regulatory T (Treg) cells in the subject.

12. The method of claim 1, wherein the application of the alternating electrical field and administration of the one or more CD137 agonists reduces expansion of Treg cells in the subject.

13. The method of claim 1, wherein the application of the alternating electrical field and administration of the one or more CD137 agonists neutralizes sCD137.

14. A method of promoting an effector T (Teff) cell immune response in a subject comprising:
b) applying an alternating electric field, at a frequency for a period of time, to a target site of the subject in need thereof; and
c) administering one or more CD137 agonists to the subject.

15. A method of reducing expansion of regulatory T (Treg) cells in a subject in need thereof
comprising:
a) applying an alternating electric field, at a frequency for a period of time, to a target site of the subject in need thereof; and
b) administering one or more CD137 agonists to the subject.

16. The method of claim 1, wherein the one or more CD137 agonists is a CD137 agonistic antibody.

17. The method of claim 16, wherein the CD137 agonistic antibody is one or more of Utomilumab (PF-05082566), ATOR 1017, AGEN2373, EU101, CTX-471, ADG106, GEN1046 (DuoBody-PD-L1×4-1BB), PRS-343 (HER2×4-1BB)/Cinrebafusp alfa, RO7122290, ADG206, LVGN6051, ABL503 or any combination thereof.

18. The method of claim 1, wherein the subject has cancer.

19. The method of claim 1, wherein the alternating electric fields are applied before, after, or simultaneously with administering the one or more CD137 agonists.

20. The method of claim 1, wherein the alternating electric field has a frequency of between 50 kHz and 1 MHz; and/or
wherein the alternating electric field has a field strength of between 0.5 and 4 V/cm RMS.
